(19) United States
(12) Patent Application Publication
(10) Pub. No.: US 2013/0287859 A1
(43) Pub. Date: Oct. 31, 2013

(54) METHOD FOR TREATING PRUPRITUS WITH CARTILAGE EXTRACT

(71) Applicant: LESCARDEN INC., New York, NY (US)

(72) Inventors: Xavier Gras Balaguer, Barcelona (ES); William Luther, Norwalk, CT (US)

(73) Assignee: LESCARDEN INC., New York, NY (US)

(21) Appl. No.: 13/830,890

(22) Filed: Mar. 14, 2013

Related U.S. Application Data
(60) Provisional application No. 61/638,568, filed on Apr. 26, 2012.

(51) Int. Cl. A61K 35/12

(52) U.S. Cl. CPC A61K 35/35
USPC 424/548

(57) ABSTRACT

Disclosed herein is a method of treating psoriatic pruritus by topical administration of a cartilage extract to the affected areas of a patient's skin and a topical composition for use in practicing the method.

### FIELD OF THE INVENTION

The present invention pertains to methods of treating pruritus associated with psoriasis in mammals by topically administering effective amounts of a cartilage extract.

### BACKGROUND OF THE INVENTION

Psoriasis is a chronic skin disease, characterized by scaling and inflammation. The disease affects 1.5 to 2 percent of the United States population, or almost 5 million people. It occurs in all age groups and about equally in men and women. Patients afflicted with psoriasis suffer discomfort, restricted motion of joints, and emotional distress. When psoriasis develops, patches of skin thicken, redden, and become covered with silvery scales, referred to as plaques. Psoriasis most often occurs on the elbow, knees, scalp, lower back, face palms, and soles of the feet. The disease also may affect the fingernails, toenails, and the soft tissue inside the mouth and genitalia.

Although psoriasis is not life threatening, the social stigma and reduction in quality of life associated with the disease are profound issues for these patients and their families. Currently, there is no long-term cure for psoriasis. Established topical anti-psoriasis therapies have been grouped into suppressive and remittive types. Suppressive therapies include coal tar preparations (natural coal tar or the distillate anthralin), topical corticosteroids, mechanical treatments to remove scale, and antimetabolites such as methotrexate. For remittive therapy, the photosensitizing, drug, psoralen, combined with long wavelength ultraviolet light (PUVA), and synthetic retinoids or coal tar derivatives also is used. While mild to moderate cases can be treated somewhat effectively, more extensive cases are difficult and tend to be resistant to either topical therapy or ultraviolet phototherapy. Moreover, systemic use of traditional anti-psoriatic drugs, or prolonged use of topical steroids, can lead to undesirable side effects or rebound worsening of psoriasis. There are new systemic immunosuppressive oral treatments that help to significantly reduce signs and symptoms of Psoriasis. However, side effects profile and cost are making them drugs for only those patients with severe disease.

Pruritus (itching) is an unpleasant sensation that causes the desire or reflex to scratch. Itch has resisted many attempts to classify it as any one type of sensory experience. Modern science has shown that itch has many similarities to pain, and while both are unpleasant sensory experiences, their behavioral response patterns are different. Pain creates a withdrawal reflex while itch leads to a scratch reflex.

Prignano et al. (Clinical Cosmetic Invest Derm 2009; 2, 9-13) reported that almost 85% of psoriatic patients suffered from pruritus. The frequency of pruritus in these patients was said to be daily and mean intensity was said to be moderate. The authors reported that almost all patients were unsatisfied with their treatment modalities for pruritus. Although the current treatments (emollients, topical steroids and calcipotriol creams) were said to partially relieve their pruritus, their effects were reported to be temporary. Alternative treatments included phototherapy with narrow band ultraviolet B radiation (nb-UVB). Nb-UVB was said to be the most effective treatment for reducing pruritus. However, narrow-band UVB can result in burning, just like sunlight and broadband UVB ND, and sometimes provokes polymorphous light eruption (PMLE). PMLE a common rash that occurs as a result of photosensitivity causes a burning sensation or itch that lasts several days. Nb-UVB can also accelerate the aging of the skin. However, once Nb-UVB treatment is discontinued, pruritus returns in almost all cases. Furthermore, the safety profile for long-term treatments with this technique is not well established.

Although a variety of over-the-counter and prescription anti-itch drugs are available they are either unsatisfactory in patient treatment or not safe for long term use. Topical anti-pruritics in the form of creams and sprays are often available over-the-counter. Oral anti-itch drugs also exist and are usually prescription drugs. The active ingredients usually belong to the following classes:
Antihistamines such as diphenhydramine (Benadryl);
Corticosteroids such as hydrocortisone topical cream;
Local anesthetics such as benzocaine topical cream (Lanacane) (sp?);
Counterirritants, such as mint oil, menthol, or camphor;
Crotamiton (trade name Euras) is an antipruritic agent available as a cream or lotion often used to treat scabies. Its mechanism of action remains unknown.

Sometimes scratching relieves isolated itches, hence the existence of devices such as the back scratcher. Often however, scratching can intensify itching and even cause further damage to the skin, dubbed the “itch-scratch-itch cycle”. The mainstay of therapy for dry skin is maintaining adequate skin moisture and topical emollients.

Prudden, J. F. (Seminars in Arthritis and Rheumatism, Vol III, No. 1, 237-321, 1974) reported treating two patients with pruritus ani by application of a 5% bovine cartilage extract preparation However, pruritus ani is not the same as the pruritus accompanying psoriatic pruritus. Pruritus ani is mediated by different chemical and cellular types than psoriatic pruritus. Furthermore psoriatic pruritus occurs in conjunction with a complex immune disease whereas pruritus ani is a localized disorder and may result from other diseases.

None of the above mentioned agents which are safe for long term use have been reported to be effective in treating patients afflicted with psoriasis who suffer from pruritus. Bigliardi, R. L. (Rev Med Suisse 2006, April 26; 2(63) 1115-6) described the diagnosis and treatment of pruritus as “ . . . still a great problem”. Several different types of pruritus were said to exist and the treatment of the different forms was said to vary. The author concluded that the diagnosis and treatment of the different forms of pruritus needed an interdisciplinary approach. Therefore, what is needed in the art are safe and effective agents for treating patients afflicted with psoriasis who suffer from pruritus.

### SUMMARY OF THE INVENTION

It has now been discovered that topical application of a cream or ointment containing between 10% and 25% by weight of cartilage extract is an effective treatment for the pruritus that accompanies psoriasis.

In one aspect, the present invention provides a method for treating pruritus in a patient afflicted with psoriasis comprising topically administering to the patient a composition comprising between about 10% and about 25% by weight of a cartilage extract.

In another aspect the invention provides a method for treating pruritus in a patient afflicted with psoriasis comprising topically administering a composition comprising 20% by weight of a cartilage extract.

In yet another aspect the invention comprises a method for treating psoriatic pruritus by topically administering an effective amount for treating psoriatic pruritus of a composition comprising 20% by weight of a bovine cartilage extract.

These and other aspects of the present invention will be apparent to those of ordinary skill in the art in light of the present description and appended claims

### DETAILED DESCRIPTION OF THE INVENTION

The following terms used herein have the meaning set forth below:

A “cartilage extract” refers to a cartilage product derived from bovine, reptilian, animal, shark and fish sources that has been digested with acid and an enzyme.

A “pharmaceutical excipient” comprises a material such as an adjuvant, a carrier, pH-adjusting and buffering agents, tonicity adjusting agents, wetting agents, preservatives, and the like.

“Pharmaceutically acceptable” refers to a non-toxic, inert, and/or biologically active composition that is physiologically compatible with humans or other mammals.

“Pharmaceutical composition” shall mean a composition comprising cartilage extract and at least one ingredient that is not cartilage extract (for example and not for limitation, a filler, dye, or a mechanism for slow release), whereby the composition is amenable to administration for a specified, efficacious outcome in a mammal (e.g., without limitation, the mammal is a human).

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Pursuant to the present invention, it has now been discovered that pruritus (itching), and especially the pruritus associated with psoriasis, can be treated by topically administering a pharmaceutical formulation comprising an amount of cartilage extract effective to treat pruritus. Prior to the present invention there were no safe and effective agents for long term treatment of this condition in patients suffering from psoriasis.

In one preferred embodiment, a pharmaceutical composition comprising between 10% and 25% bovine cartilage extract is administered to patients afflicted with pruritus associated with psoriasis. In another preferred embodiment a composition comprising between 15 and 20% by weight of bovine cartilage is administered to patients afflicted with pruritus associated with psoriasis. In yet another preferred embodiment, a pharmaceutical composition comprising an ointment containing 20% by weight of bovine cartilage extract is administered topically to patients afflicted with pruritus and psoriasis. The amount of cartilage extract to be administered and the frequency of administration can be determined by a physician based on the severity of the pruritus. Depending on severity of disease, the topical extract is applied at least once per day, but preferably two or three times per day directly on the surface of the lesion(s) that are the source of the itching sensation. If pruritus improves or is less severe application once each day is adequate. Thus, the method of the present invention comprises topically administering a pharmaceutical formulation containing an effective amount for treating psoriatic pruritus of a cartilage extract, preferably bovine cartilage extract, to treat pruritus associated with psoriasis.

The cartilage extracts used in the compositions of the present invention are known in the art and have been described in Balassa et al. U.S. Pat. Nos. 3,400,199, 3,476,855, 3,478,146 and 3,772,432, the entire disclosures of which are incorporated herein by reference. As set forth in the Balassa et al patents above, the cartilage powder used in preparing the pharmaceutical composition of the present invention may be derived from young cartilage, i.e., from young animals (e.g. calves) or young or newly regenerated cartilage from older animals as reptiles or from species such as fish or shark in which the cartilage remains young eternally. Where age is the criteria for defining “young” the cartilage is preferably derived from animals not over six months old. However, cartilage powder derived from the cartilage of older animals may also be employed. The preferred embodiment of the invention employs cartilage from cows that are less than 3 years old. The preferred source of cartilage for use in the present invention is bovine cartilage that is free from contamination by bovine spongiform encephalitis (BSE).

The cartilage may be prepared by any suitable means to result in a product which is essentially pure cartilage substantially free from adhering tissue which may have been removed by acid-pepsin or other suitable enzyme treatment, with or without mechanical assistance or otherwise. See, for example, the procedures outlined in the Balassa et al. patents identified above which may be employed in formulating the topical cartilage compositions of the present invention.

To be effective in treating psoriatic pruritus, pharmaceutical formulations for use in the present invention contain between about 10% and about 25% by weight cartilage extract and more preferably between about 15% and 25% by weight of cartilage extract and in an especially preferred embodiment contain 20% by weight of cartilage extract.

Pharmaceutical formulations containing cartilage extract adapted for topical administration according to the present invention may be formulated as ointments, creams, suspensions, lotions, powders, solutions, pastes, gels, sprays, aerosols or oils. The topical formulations of the present invention may contain a variety of ingredients and pharmaceutical excipients that are customarily employed in preparing topical creams, ointments, lotions and gels. Non-limiting examples of such ingredients are Ammonium Lauryl Sulfate, Ammonium Laureth Sulfate, Sodium Lauryl Sulfate, Sodium Laureth Sulfate, Amphoteric Surfactants such as cocoamphoglycinate, cocoamidopropyl, Betaines and the like, amine oxides, such as cocoamine oxide and the like, cellulose and cationic cellulose like Polyquat 10, Guar Gum and Cationic Guar Gum, UV absorbing compounds such as Benzophenone 3 and Octylmethoxycinnamate, Silicone fluids (Cyclomethicone), modified silicone fluids (Amodimethicone), Botanical extracts, Fatty Esters and triglycerides such as Octyl Stearate and Wheat Germ Oil, Alkoxylated Glycerides such as PEG-30 Glyceryl Cocoate, Alkoxylated Sorbitan Esters such as Polysorbate 20, Carboxylated surfactants such as Trideceth-10 Carboxylate, Lanolin and Lanolin quats, and Petrolatum. The pharmaceutical formulations according to the present invention may include other pharmaceutically acceptable ingredients including, without limitation, and in any pharmaceutically acceptable combination, including by way of non-limiting example, keratolytic or peeling agents (e.g. salicylic acid), moisturizers (e.g. petrolatum), anti-inflammatory agents (e.g. Calcipotriene, a form of synthetic vitamin D3 combined with the steroid betamethasone dipropionate), antioxidants (e.g. butylated hydroxyanisole, butylated hydroxytoluene, ascorbic acid, edetic acid, sodium edetate), chelators (e.g. EDTA), retinoids (e.g. Tazarotene), skin protectants (e.g. petrolatum), and vitamins (such as vitamins A, D and Calcipotriene (synthetic vitamin D3).

The method of the present invention typically involves administering the pharmaceutical formulation of the invention containing an effective amount of cartilage extract to treat psoriatic pruritus to the psoriatic lesions on the patient's skin. In many instances this treatment not only relieves the patient's pruritus but also reduces or eliminates the patient's psoriasis.

In practicing the present invention, the cartilage extract containing formulation is topically applied (for example, in the form of a cream, salve, ointment or gel) directly to the psoriatic lesions in a sufficient amount to cover the lesion surface. The topical formulation is applied at least once daily and can be repeatedly applied during the day if the itching sensation returns or persists. The number of applications required each day will vary from patient to patient depending on the extent and severity of the psoriatic lesions with which the patient is afflicted. In some instances, it may be desirable to employ an occlusive dressing to hold the topical formulation in position. Suitable occlusive dressings can be bandages, sterile gauze pads, or plastic films or tapes.

The present invention is based on the finding that topical administration of a topical pharmaceutical formulation containing 10-25% by weight of bovine cartilage extract to patients afflicted with psoriasis and simultaneously suffering from pruritus successfully treated the patients' pruritus. Surprisingly, the treatment was effective in alleviating the itch sensation associated with psoriasis and psoriatic peaques or scales for almost all of the patients in the study. Prior to the present invention there were no known safe and effective long term treatments for this aspect of the disease. A clinical trial of the topical formulation of the invention is described below.

In Example 4, twelve patients afflicted with psoriatic pruritus were studied. All of the patients participating in the study discontinued any topical treatment 1 month before starting the trial. This was considered a washout period to eliminate any influence on the study results by medications or treatments previously administered to the patient. Patients were evaluated one month after beginning daily topical application of the cartilage extract formulation on the psoriatic lesions selected for treatment.

Eleven patients experienced a significant improvement of their psoriatic lesions and their pruritus. The last patient experienced a mild improvement only of his lesion but not his pruritus. This improvement started a few hours after the first application of the topical cartilage composition. Eight treated patients asked to continue receiving treatment with the topical cartilage composition after conclusion of the study in order to continue alleviation of the itching sensation accompanying their psoriasis. No side effects were reported.

The present invention is described below in working examples which are intended to further illustrate the invention without limiting the scope thereof.

### Example 1

### Bovine Cartilage Extract Production

Bovine cartilage extract for use in the present invention was produced as follows.

Cartilage containing tissue (from bovine animals) was collected only from New Zealand cows. New Zealand animals are certified by the World Health Organization to be free of bovine spongiform encephalopathy (“BSE”). Further, all cartilage containing tissue was harvested from proven healthy animals before being pooled for production purposes.

Bovine tracheas were obtained and digested with enzymes such as pepsin in an acid solution (e.g. acetic acid) for more than four hours to remove the soft tissue. After rinsing, the cartilage was ground and extracted for several hours with an organic solvent (e.g. acetone) and then dried in a vacuum. The dried granules were ground to a powder having an average particle size of about 35 microns.

The dried cartilage powder of Example 1 was admixed with the constituents described in Example 3 below to create a topical formulation suitable for administration to human patients.

### Example 2

### Topical Pharmaceutical Formulation Containing Bovine Cartilage Extract

Cartilage Ointment Formulations

The following table sets forth the range of ingredients (and their percentage composition) that can be used in preparing topical cartilage composition for use in the present invention.

TABLE 1

| INGREDIENT | | |
|---|---|---|
| NAME | WEIGHT PERCENT | FUNCTION |
| Petrolatum | 30.01-100.00 | Skin protectant |
| Cartilage Powder | 20.00 | Skin conditioning |
| | | agent |
| Isopropyl Palmitate | 3.01-10.00 | Emollient |
| Caprylic/Capric Triglyceride | 3.01-10.00 | Emollient |
| Peg-30 Dipolyhydroxystearate | 3.01-10.00 | Surfactant |
| Polyethylene (MW??) | 1.01-3.00 | Stability |
| | | enhancement |
| | | agent |
| Beeswax | 0.3-1.00 | Skin conditioning |
| | | agent |
| Paraffin | 0.3-1.00 | Viscosity |
| | | increasing |
| | | agent |
| Benzyl alcohol | 0.3-1.00 | Preservative |

### Example 3

### Cartilage Extract Ointment

Using the bovine cartilage extract of Example 1, the following ointment formulation was prepared.

TABLE 2

| INGREDIENT | WEIGHT | |
|---|---|---|
| NAME | PERCENT | FUNCTION |
| Petrolatum | 54.50 | Skin protectant |
| Cartilage Powder | 20.00 | Skin conditioning agent |
| Isopropyl Palmitate | 10.00 | Emollient |
| Caprylic/Capric Triglyceride | 5.00 | Emollient |
| Peg-30 Dipolyhydroxystearate | 5.00 | Surfactant |
| Polyethylene | 3.00 | Stability enhancement agent |
| Beeswax | 1.00 | Skin conditioning agent |
| Paraffin | 1.00 | Viscosity increasing agent |
| Benzyl alcohol | 0.50 | Preservative |

The formulation was topically administered to twelve patients afflicted with pruritus associated with psoriasis as set forth in Example 4 below.

### Example 4

### Trial of Cartilage Extract Formulation in Psioriatic Pruritus Patients

### Basic Demographic Data for Trial

12 Patients of both sexes from an ambulatory clinic with mild/moderate psoriasis were chosen to be treated with the formulation of Example 3.
Sex distribution: 8 females and 4 males
Age range: 24-68 years
Duration of the diseases in each patient: average 20 years
All patients had been treated with several topical and systemic anti-psoriatic and anti-pruritic products and drugs without success. In general the results of such treatment were unsatisfactory to the patients.
Washout period: 1 month. During the washout period, all patients discontinued use of any treatment or pharmaceutical agents for their psoriasis or pruritus.
Clinical data from all patients was recorded.
All patients acknowledged their participation in an experimental trial.

### Instructions

Patients were instructed to apply the cartilage containing ointment of Example 3 once a day covering the psoriatic lesions selected for evaluation.
Treatment duration: 4-6 weeks
All patients were asked to report any unexpected adverse reactions and/or side effects and to immediately stop the treatment and contact the investigator in the event of such reactions.
Patients' impressions were recorded.
Patients were evaluated thirty (30) days after beginning treatment.
   Pruritus was evaluated according to the following score:
   4=severe/intense
   3=moderate
   2=mild
   1=very mild
   0=not present
      The puritus score for each patient (before and after treatment) is set forth in Table 3.

Results

TABLE 3

| | | | Pruritus Score | Pruritus Score |
|---|---|---|---|---|
| Patient | Age | Sex | Before Treatment | After Treatment |
| CBY | 26 | Female | 2 | 0 |
| ENG | 50 | Female | 4 | 1 |
| JBP | 46 | Male | 2 | 0 |
| JGD | 44 | Male | 3 | 2 |
| JPL | 42 | Male | 4 | 2 |
| MNL | 60 | Male | 3 | 0 |
| MPT | 56 | Woman | 4 | 0 |
| MTMP | 29 | Woman | 3 | 1 |
| PNG | 51 | Woman | 3 | 1 |
| PPL | 61 | Woman | 3 | 0 |
| RFG | 58 | Woman | 4 | 0 |
| ARL | 28 | Woman | 1 | 0 |

Results

A significant reduction in the itching sensation was experienced by all patients treated. This effect appeared few hours after the first application of the cartilage product and lasted almost a full day. After several daily applications, the itching sensation disappeared in almost all patients.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all values are approximate, and are provided for description. Patents, patent applications, publications, product descriptions, and protocols are cited throughout this application, the disclosures of which are incorporated herein by reference in their entireties for all purposes.

What is claimed is:

1. A method for treating pruritus in a patient suffering from psoriasis comprising topically administering a composition comprising between about 10% and about 25% of a cartilage extract.

2. The method of claim 1 wherein the cartilage is bovine origin cartilage.

3. The method of claim 2 wherein the composition contains between about 15% and about 25% cartilage extract by weight.

4. The method of claim 3 wherein the composition comprises a cream or an ointment.

5. The method of claim 3 which comprises applying the cartilage composition at least once a day.

6. The method of claim 2 wherein the composition contains 20% by weight of cartilage extract.

7. The method of claim 6 wherein the cartilage extract is free from BSE.

8. The method of claim 3 which comprises covering the composition with an occlusive dressing after administration to the patient.

9. The method of claim 3 wherein the composition comprises an emollient.

10. The method of claim 4 which comprises administering the composition by depositing it directly on the psoriatic scales on the patient's skin.

11. A topical composition comprising between 10% and 25% by weight of a cartilage extract in a pharmaceutically acceptable cream or ointment.
